SEQUENCE LISTING (12 sequences)

SEQ ID NO: 1 - DNA, 21 bp, Artificial Sequence
  This sequence is synthesized.
  misc_feature: delF
ttagaggaattagacatctct

SEQ ID NO: 2 - DNA, 17 bp, Artificial Sequence
  This sequence is synthesized.
  misc_feature: delR
tgaaagatttgagccca

SEQ ID NO: 3 - DNA, 20 bp, Artificial Sequence
  This sequence is synthesized.
  misc_feature: TransvF
tcaaatcaggagtctgacga

SEQ ID NO: 4 - DNA, 20 bp, Artificial Sequence
  This sequence is synthesized.
  misc_feature: TransvR
aggtgcacagaaaccgatta

SEQ ID NO: 5 - DNA, 118 bp, Artificial Sequence
  This sequence is synthesized.
  misc_feature: Del-target (wt)
ttagaggaattagacatctctaaaaattccctaagtttcttgccttctggagtttttgatggtatgcctccaaatctaaagaatctctctttggccaaaaatgggctcaaatctttca

SEQ ID NO: 6 - DNA, 114 bp, Artificial Sequence
  This sequence is synthesized.
  misc_feature: Del-target (m)
ttagaggaattagacatctctaaaaattccctaagtttctttctggagtttttgatggtatgcctccaaatctaaagaatctctctttggccaaaaatgggctcaaatctttca

SEQ ID NO: 7 - DNA, 167 bp, Artificial Sequence
  This sequence is synthesized.
  misc_feature: Transv-target (wt)
tcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacct

SEQ ID NO: 8 - DNA, 167 bp, Artificial Sequence
  This sequence is synthesized.
  misc_feature: Transv-target
tcaaatcaggagtctgacgaagtattttctacaagatgccttccagtttcgatatctggatctcagctcaaataaaatccagatgatccaaaagaccagcttcccagaaaatgtcctcaacaatctgaagatgttgcttttgcatcataatcggtttctgtgcacct

SEQ ID NO: 9 - DNA, 23 bp, Artificial Sequence
  This sequence is synthesized.
  misc_feature: delp
taagtttcttgccttctggagtt

SEQ ID NO: 10 - DNA, 29 bp, Artificial Sequence
  This sequence is synthesized.
  misc_feature: delPm
attccctaagtttctttctggagtttttg

SEQ ID NO: 11 - DNA, 24 bp, Artificial Sequence
  This sequence is synthesized.
  misc_feature: transvP
tgccttccagttgcgatatctgga

SEQ ID NO: 12 - DNA, 24 bp, Artificial Sequence
  This sequence is synthesized.
  misc_feature: transvPm
tgccttccagtttcgatatctgga

(19) United States
(12) Patent Application Publication
(10) Pub. No.: US 2024/0318238 A1
(43) Pub. Date: Sep. 26, 2024

(54) METHOD OF USING REAL-TIME PCR DEVICE IN A NEW DIAGNOSTIC FIELD AND DIAGNOSTIC KIT DEVELOPED TO USE IN REAL-TIME PCR DEVICE FOR THIS NEW DIAGNOSTIC FIELD

(71) Applicant: Cagcil YETIM, Istanbul (TR)

(72) Inventor: Cagcil YETIM, Istanbul (TR)

(21) Appl. No.: 18/034,388

(22) PCT Filed: Oct. 21, 2021
(86) PCT No.: PCT/TR2021/051078

§ 371 Date: Apr. 28, 2023

(30) Foreign Application Priority Data
Oct. 28, 2020 (TR) 2020/17266
Dec. 17, 2020 (TR) 2020/20743
Oct. 20, 2021 (TR) 2021/016366

(51) Int. Cl. C12Q 1/6858

(52) U.S. Cl. CPC C12Q 1/6858

(57) ABSTRACT

A method of using a real-time PCR device for the detection of normal and rare variants of TLR7 gene and moreover a diagnosis kit developed for use in real-time PCR device for this new diagnosis field and developed for detection of normal and rare variants of TLR7 gene is provided.

### CROSS REFERENCE TO RELATED APPLICATIONS

This application is the national stage entry of International Application No. PCT/TR2021/051078 filed Oct. 21, 2021, and which is based upon and claims priority to Turkish Patent Application No. 2020/17266 filed Oct. 28, 2020, Turkish Patent Application No. 2020/20743 filed Dec. 17, 2020, and Turkish Patent Application No. 2021/016366 filed Oct. 20, 2021, the entire contents of which are incorporated by reference herein.

### SEQUENCE LISTING

The instant application contains a Sequence Listing which has been submitted in ASCII format via EFS-Web and is hereby incorporated by reference in its entirety. Said ASCII copy is named GBERD057_ST25_Sequence_Listing.txt, created on Apr. 27, 2023, and is 3,841 bytes in size.

### TECHNICAL FIELD

The present invention relates to the usage of real-time PCR device in a new and different diagnosis and moreover a diagnosis kit developed for use in real-time PCR device for this new diagnosis field.

### BACKGROUND

Infectious disease is a public health problem which affects all humanity. Infectious diseases affect societies socially, economically and culturally in periods where infectious diseases occur in a dense manner.

In viral infections, natural immunity response is triggered firstly by lung epithelial cells, alveolar macrophages and neutrophils. In the next phase, T and B lymphocytes become active as a result of activation of the gained immunity response. The viral particles which comprise single-stranded RNA (ssRNA) behave like pathogen-associated molecular patterns (PAMPs) and are recognized by Toll-like receptor 7 (TLR7) provided on the surface of macrophages and dendritic cells and provide beginning of natural immunity response. TLR7 activates various signal pathways and transcription factors and leads to synthesis of monocyte chemo-attractant protein-1 (MCP-1) of primary cytokines like IL-1 and IL-6 related to inflammation, macrophage inflammatory protein 1 alpha (MIP-1A), tumor necrosis factor (TNF-) and antiviral Type 1 interferon (IFN1).

Rare variants of X chromosomal TLR7 gene are associated with deteriorated type I and II IFN responses, and they lead to insufficiency of the immune system in the first defense response against the viral infection.

Therefore, detection of rare variants of TLR7 gene in a rapid manner is important. In the present system, the rare variants, which affect TLR 7 gene, can be detected by means of Sanger arraying method, however, featured genetic laboratories are needed for this method, and this method gives result in longer duration for realizing diagnosis.

As a result, because of the abovementioned problems, an improvement is required in the related technical field.

### SUMMARY

The present invention relates to the usage of real-time PCR device in the detection of normal TLR7 gene and TLR7 gene mutations, and moreover a diagnosis kit developed for use in real-time PCR device for the detection of normal TLR7 gene and also TLR7 gene mutations (variants which are rare and which affect sufficient functioning of the TLR7 gene), for eliminating the abovementioned disadvantages and for bringing new advantages to the related technical field.

The main object of the present invention is to provide usage of real-time PCR device and a new diagnosis kit, which is to be used in real-time PCR device, in detection of normal TLR7 gene and also variants of TLR7 gene, which are rare and which affect sufficient functioning of the TLR7 gene.

### DETAILED DESCRIPTION OF THE INVENTION

In this detailed description, the subject matter test kit and detection method is explained with references to examples without forming any restrictive effect only in order to make the subject more understandable.

The present invention relates to a diagnosis kit developed for use in real-time PCR device for detection of normal TLR7 gene and variants of TLR7 gene (mutations), wherein the subject matter diagnosis kit comprises a reaction mixture comprising at least one or some or all of delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6), Transv-target (wt) (SEQ ID NO: 7), Transv-target(m) (SEQ ID NO: 8) primers and delp (SEQ ID NO: 9) delPm (SEQ ID NO: 10) transvP (SEQ ID NO: 11) transvPm (SEQ ID NO: 12) probes.

The present invention relates to a diagnosis kit developed for use in real-time PCR device for detection of normal TLR7 gene and variants of TLR7 gene (mutations), wherein the subject matter diagnosis kit comprises a reaction mixture comprising at least one or some or all of delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6), Transv-target(wt) (SEQ ID NO: 7), Transv-target(m) (SEQ ID NO: 8) primers.

In an embodiment, the reaction mixture which exists in the subject matter diagnosis kit comprises a mixture of delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6), Transv-target(wt) (SEQ ID NO: 7), Transv-target(m) (SEQ ID NO: 8) primers.

In an embodiment, the reaction mixture which exists in the subject matter diagnosis kit comprises a mixture of delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6), Transv-target(wt) (SEQ ID NO: 7), Transv-target(m) (SEQ ID NO: 8) primers and delp (SEQ ID NO: 9), delPm (SEQ ID NO: 10), transvP (SEQ ID NO: 11), transvPm (SEQ ID NO: 12) probes.

In an embodiment, the reaction mixture which exists in the subject matter diagnosis kit comprises a mixture of delF (SEQ ID NO: 1), delR (SEQ ID NO: 2), TransvF (SEQ ID NO: 3), TransvR (SEQ ID NO: 4) primers and delp (SEQ ID NO: 9), delPm(SEQ ID No:10), transvP (SEQ ID NO: 11), transvPm (SEQ ID NO: 12) probes.

In an embodiment, the reaction mixture comprises a mixture of delp (SEQ ID NO: 9), delPm (SEQ IDNO: 10), transvP (SEQ ID NO: 11), transvPm (SEQ ID NO: 12) probes.

In an embodiment, the reaction mixture comprises delF (SEQ ID NO: 1) and delR (SEQ ID NO: 2) primers.

In an embodiment, the reaction mixture comprises delF (SEQ ID NO: 1), delR (SEQ ID NO: 2) Del-target(wt) (SEQ ID NO: 5), Del-target(m) (SEQ ID NO: 6) primers.

In an embodiment, the reaction mixture comprises TransvF (SEQ ID NO: 3), TransvR (SEQ ID NO: 4) primers.

In an embodiment, the reaction mixture comprises TransvF (SEQ ID NO: 3), TransvR (SEQ ID NO: 4), Transv-target(wt) (SEQ ID NO: 7), Transv-target(m) (SEQ ID NO: 8) primers.

In an embodiment, the reaction mixture comprises Del-target(wt) (SEQ ID NO: 5), Del-target(m) (SEQ ID NO: 6) primers.

In an embodiment, the reaction mixture comprises Transv-target(wt) (SEQ ID NO: 7), Transv-target(m) (SEQ ID NO: 8) primers.

In an embodiment, the reaction mixture comprises delF (SEQ ID NO: 1), delR (SEQ ID NO: 2), TransvF (SEQ ID No:3), TransvR (SEQ ID NO: 4) primers.

In an embodiment, the subject matter diagnosis kit can comprise the mixture of at least one or some or all of delF (SEQ ID NO: 1), delR (SEQ ID NO: 2), TransvF (SEQ ID NO: 3), TransvR (SEQ ID NO: 4), Del-target(wt)(SEQ ID NO: 5), Del-target(m) (SEQ ID NO: 6), Transv-target(wt) (SEQ ID NO: 7), Transv-target(m) (SEQ ID NO: 8) primers and additionally, reaction buffer and/or sterile water and/or any DNA polymerase enzyme and/or Mg+2 ion, for instance, MgCl2 and/or more different enzyme or enzymes and/or more different reaction buffer and/or additional substance mixtures.

The nucleic acid arrays of the delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6) Transv-target(wt) (SEQ ID NO: 7), Transv-target(m) (SEQ ID NO: 8) primers which are mentioned in the application have been given in Table 1 provided in the examples section and in the sequence list enclosed to this specification.

The nucleic acid arrays of the delp (SEQ ID NO: 9), delPm (SEQ ID NO: 10), transvP (SEQ ID NO: 11), transvPm (SEQ ID NO: 12), Del-target(wt) probes which are mentioned in the application have been given in Table 2 provided in the examples section and in the sequence list enclosed to this specification.

The term deIF which exists in the specification describes the primer which has 5′-TTAGAGGAATTAGACATCTCT-3′ sequence array, and this sequence array is named briefly as SEQ ID NO: 1 in the specification.

The term deIR which exists in the specification describes the primer which has 5′-TGA AAG ATT TGA GCC CA-3′ sequence array, and this sequence array is named briefly as SEQ ID NO: 2 in the specification.

The term TransvF which exists in the specification describes the primer which has 5′-TCA AAT CAG GAG TCT GAC GA-3′ sequence array, and this sequence array is named briefly as SEQ ID NO: 3 in the specification.

The term TransvR which exists in the specification describes the primer which has 5′-AGG TGC ACA GAA ACC GAT TA-3′ sequence array, and this sequence array is named briefly as SEQ ID NO: 4 in the specification.

The term Del-target (wt) which exists in the specification describes the primer which has TTAGAGGAATTAGACATCTCTAAAAATTCCCTAAGTTTCTTGCCTTCTGGAGTTTT TGATGGTATGCCTCCAAATCTAAAGAATCTCTCTTTGGCCAAAAATGGGCTCAAA TCTTTCA sequence array, and this sequence array is named briefly as SEQ ID NO: 5 in the specification.

The term Del-target (m) which exists in the specification describes the primer which has TTAGAGGAATTAGACATCTCTAAAAATTCCCTAAGTTTCTTTCTGGAGTTTTTGAT GGTATGCCTCCAAATCTAAAGAATCTCTCTTTGGCCAAAAATGGGCTCAAATCTT TCA sequence array, and this sequence array is named briefly as SEQ ID NO: 6 in the specification.

The term Transv-target (wt) which exists in the specification describes the primer which has TCAAATCAGGAGTCTGACGAAGTATTTTCTACAAGATGCCTTCCAGTTGCGATAT CTGGATCTCAGCTCAAATAAAATCCAGATGATCCAAAAGACCAGCTTCCCAAAAA TGTCCTCAACAATCTGAAGATGTTGCTTTTGCATCATAATCGGTTTCTGTGCACCT sequence array, and this sequence array is named briefly as SEQ ID NO: 7 in the specification.

The term Transv-target (m) which exists in the specification describes the primer which has TCAAATCAGGAGTCTGACGAAGTATTTTCTACAAGATGCCTTCCAGTTTCGATATC TGGATCTCAGCTCAAATAAAATCCAGATGATCCAAAAGACCAGCTTCCCAGAAA ATGTCCTCAACAATCTGAAGATGTTGCTTTTGCATCATAATCGGTTTCTGTGCACC T sequence array, and this sequence array is named briefly as SEQ ID NO: 8 in the specification.

The term delp which exists in the specification describes the probe which has FAM-TAAGTTTCTTGCCTTCTGGAGTT sequence array, and this sequence array is named briefly as SEQ ID NO: 9 in the specification.

The term delPm which exists in the specification describes the probe which has HEX-ATTCCCTAAGTTTCTTTCTGGAGTTTTTG sequence array, and this sequence array is named briefly as SEQ ID NO: 10 in the specification.

The term transvP which exists in the specification describes the probe which has Cy5-TGCCTTCCAGTTGCGATATCTGGA-BHQ2 sequence array, and this sequence array is named briefly as SEQ ID NO: 11 in the specification.

The term transvPm which exists in the specification describes the probe which has Cy5,5-TGCCTTCCAGTTTCGATATCTGGA-BHQ2 sequence array, and this sequence array is named briefly as SEQ ID NO: 12 in the specification.

The subject matter test kit has a reaction mixture comprising the mixture of at least one, some or all of the delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6) Transv-target(wt) (SEQ ID NO: 7), Transv-target(m) (SEQ ID NO: 8) primers.

The subject matter test kit has a reaction mixture comprising the mixture of at least one, some or all of the (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6) Transv-target(wt) (SEQ ID NO: 7), Transv-target(m) (SEQ ID NO: 8) primers and delp (SEQ ID NO: 9), delPm (SEQ ID NO: 10), transvP (SEQ ID NO: 11), transvPm (SEQ ID NO: 12) probes.

The delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6) Transv-target(wt) (SEQ ID NO: 7), Transv-target(m) (SEQ ID NO: 8) primers which exist in the reaction mixture are specific primers which detect of normal TLR7 gene and variants of TLR7 gene (mutations). They realize detection of the mutant variants. As in the general operation of real-time PCR technology, when the primers which exist in the reaction mixture reach a suitable temperature in the cycle after enzyme activation and incubation in the device, the primers bind to the specific complementary regions and provide operation of DNA polymerase enzyme and DNA amplification in this region. Even if DNA amount, which increases exponentially during cycles, is low, specific variants can be displayed such that signal can be received. The primers, designed for the subject matter, serve this purpose, and they provide detection of the variants, of which the presence is searched, by means of fluorescent signals. In the invention, DNA oligo-nucleotide array is provided which is called 2 sets of primers. These are designed so as to be complementary for the related arrays for detecting the variants, which may lead to RNA transcription of TLR7 without function, and the wild type version thereof. These sets are independent from each other, however, they have been designed as two each primers according to one each target augmentation. One of the sets has been prepared for deletion variant detection and one of the sets has been prepared for transversion variant detection.

Wild type TLR7 detection is also realized by means of melting curve analysis and thus, inhibition control is also made. The control of operation of the test kit is realized by means of positive-negative controls.

Moreover, another version of the present invention functions as follows: As in the general operation of real-time PCR technology, when the primers which exist in the reaction mix arrive at suitable temperature in the cycle after enzyme activation and incubation in the device, they bind to the specific complementary regions and provide operation of the DNA polymerase enzyme and DNA amplification in this region. Even if the DNA amount which increases exponentially during cycles is at low proportion, it provides displaying of specific variants so as to be able to receive signal. The primers, designed particularly in the invention, serve this purpose and provide detection of the variants, the presence of which is searched, by means of fluorescent signals. In the invention, there are 3 sets of DNA oligo-nucleotide arrays which are called primer-probe. These have been designed so as to be complementary for the related arrays in order to detect variants, which may lead to RNA transcription of TLR7 without function, and the wild type version thereof. These sets are independent from each other, however, they have been designed in accordance with one each target augmentations as 2 each primers and 1 each probes. One of the sets has been prepared for the deletion detection, one of the sets has been prepared for transversion detection and the other one has been prepared for wild type variant detection.

Reaction mix: TLR7 comprises specific primer mixture which detects mutant variants. It makes detection of mutant variants.

Internal control: It realizes normal TLR7 detection, it makes inhibition control.

Positive-negative controls: It makes the control of whether the kit operates in a regular manner or not.

### Primers and Probes:

Primers and Taqqman probes have been designed for use in real-time PCR.

By means of the present invention, treatment management or protective health service shall be realized in a more effective manner by means of detection of this variant, in accordance with the information which indicates that the progress of the ssRNA virus disease in some individuals is much heavier when compared with the other individuals, particularly in COVID-19. The advantage and benefit provided by the present invention are that real-time pcr method is used and by this way, more rapid diagnosis is realized by means of easily accessible and easy method in most laboratories where real-time pcr device exists.

Another advantage of the present invention is that the present invention provides rapid and exact variant detection in the in vitro medium. This advantage is provided by specific and rapid response characteristic of the real-time PCR method, and the test can be realized by means of material like venous blood which can be easily obtained and in a manner which can be additional to routine blood tests.

By the specific detection of individuals for clinical progress of the ss RNA virus sourced diseases and the specific determination about the degree of immune response of the body for each individual, before a disease factor is encountered with the screening and detection of people who are likely to show a severe clinical progress, it will be possible for such people to benefit from preventive health services and also by this screening method in case of disease situation it can be possible for them to obtain treatment protocols early and effectively.

In one perspective, the present invention relates to the usage of delF (SEQ ID NO: 1), delR (SEQ ID NO: 2), TransvF (SEQ ID NO: 3), TransvR (SEQ ID NO: 4) primers in a method to be used in prevention of severe clinical progress of Covid-19 infection.

In one perspective, the present invention relates to the usage of delF (SEQ ID NO: 1), delR (SEQ ID NO: 2), TransvF (SEQ ID NO: 3), TransvR (SEQ ID NO: 4), Del-target(wt)(SEQ ID NO: 5), Del-target(m) (SEQ ID NO: 6) Transv-target(wt) (SEQ ID NO: 7) Transv-target(m) (SEQ ID NO: 8) primers in a method to be used in prevention of severe clinical progress of Covid-19 infection.

In one perspective, the present invention relates to the usage of delF (SEQ ID NO: 1), delR (SEQ ID NO: 2), TransvF (SEQ IDNO: 3), TransvR (SEQ ID NO: 4), Del-target(wt) (SEQ ID NO: 5), Del-target(m) (SEQ ID NO: 6) Transv-target(wt) (SEQ ID NO: 7) Transv-target(m) (SEQ ID NO: 8) primers and delp (SEQ ID NO: 9), delPm (SEQ ID NO: 10), transvP (SEQ ID NO: 11), transvPm (SEQ ID NO: 12) probes in a method to be used in prevention of severe clinical progress of Covid-19 infection.

In one perspective, the present invention relates to the usage of delF (SEQ ID NO: 1), delR (SEQ ID NO: 2), TransvF (SEQ ID NO: 3), TransvR (SEQ ID NO: 4) primers and delp (SEQ ID NO: 9), delPm (SEQ ID NO: 10), transvP(SEQ ID NO: 11), transvPm (SEQ ID NO: 12) probes in a method to be used in prevention of severe clinical progress of Covid-19 infection.

In one perspective, the present invention relates to the usage of delp (SEQ ID NO: 9), delPm (SEQ ID NO: 10), transvP(SEQ ID NO: 11), transvPm (SEQ ID NO: 12) probes in a method to be used in prevention of severe clinical progress of Covid-19 infection.

In one perspective, the present invention relates to the usage of delF (SEQ IDNO: 1), delR (SEQ ID NO: 2) in a method to be used in prevention of severe clinical progress of Covid-19 infection.

In one perspective, the present invention relates to the usage of TransvF (SEQ ID NO: 3), TransvR (SEQ ID NO: 4) primers in a method to be used in prevention of severe clinical progress of Covid-19 infection.

In one perspective, the present invention relates to the usage of delF (SEQ ID NO: 1), delR (SEQ ID NO: 2) Del-target(wt)SEQ ID NO: 5), Del-target(m) (SEQ ID NO: 6) primers in a method to be used in prevention of severe clinical progress of Covid-19 infection.

In one perspective, the present invention relates to the usage of TransvF (SEQ ID NO: 3), TransvR (SEQ ID NO: 4), Transv-target(wt) (SEQ ID No:7), Transv-target(m) (SEQ IDNO: 8) primers in a method to be used in prevention of severe clinical progress of Covid-19 infection.

In an embodiment the usage of one or some or all of the delF (SEQ ID No:1), delR (SEQ ID NO: 2), TransvF (SEQ ID NO: 3), TransvR (SEQ ID NO: 4) primers in a method to be used in detection of TLR7 gene mutation.

In an embodiment the usage of one or some or all of the delF (SEQ ID NO: 1), delR (SEQ IDNO: 2), TransvF (SEQ ID NO: 3), TransvR (SEQ ID NO: 4), Del-target(wt) (SEQ ID NO: 5), Del-target(m) (SEQ ID NO: 6) Transv-target(wt) (SEQ ID NO: 7) Transv-target(m) (SEQ ID NO: 8) primers and/or delp (SEQ ID NO: 9), delPm (SEQ ID NO: 10), transvP (SEQ ID NO: 11), transvPm (SEQ ID NO: 12) probes in a method to be used in detection of normal TLR7 gene and variants of TLR7 gene (mutations).

In an embodiment the usage of one or some or all of the delF (SEQ ID NO: 1), delR (SEQ ID NO: 2), TransvF (SEQ ID NO: 3), TransvR (SEQ ID NO: 4) primers and/or delp (SEQ ID NO: 9), delPm (SEQ ID NO: 10), transvP (SEQ ID NO: 11), transvPm (SEQ ID NO: 12) probes in a method to be used in detection of TLR7 gene mutations.

In an embodiment the usage of one or some or all of the delp (SEQ ID NO: 9), delPm (SEQ IDNO: 10), transvP (SEQ ID NO: 11), transvPm (SEQ ID NO: 12) probes in a method to be used in detection of TLR7 gene mutations.

In an embodiment the usage of one or some or all of the Del-target(wt) (SEQ ID NO: 5), Del-target(m) (SEQ ID NO: 6) Transv-target(wt) (SEQ ID NO: 7) Transv-target(m) (SEQ ID NO: 8) primers and/or delp (SEQ ID NO: 9), delPm (SEQ ID No:10), transvP (SEQ ID NO: 11), transvPm (SEQ ID NO: 12) probes in a method to be used in detection of normal TLR7 gene and TLR7 gene mutations.

In an embodiment the usage of delF (SEQ ID NO: 1), delR (SEQ ID NO: 2) primers in a method to be used in detection of TLR7 gene mutations.

In an embodiment the usage of TransvF (SEQ ID NO: 3), TransvR (SEQ ID NO: 4) primers in a method to be used in detection of TLR7 gene mutations.

In an embodiment the usage of delF (SEQ ID NO: 1), delR (SEQ ID NO: 2) Del-target(wt) (SEQ ID NO: 5), Del-target(m) (SEQ ID NO: 6) primers in a method to be used in detection of normal TLR7 gene and variants of TLR7 gene (mutations).

In an embodiment the usage of TransvF (SEQ ID NO: 3), TransvR (SEQ ID NO: 4) Transv-target(wt) (SEQ ID NO: 7) Transv-target(m) (SEQ ID NO: 8) primers in a method to be used in detection of normal TLR7 gene and variants of TLR7 gene (mutations).

In an embodiment the usage of one or some or all of the delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6) Transv-target(wt) (SEQ ID NO: 7), Transv-target(m) (SEQ ID NO: 8) primers in a method to be used in detection of normal TLR7 gene and variants of TLR7 gene (mutations).

In an embodiment a method for the detection of normal TLR7 gene and TLR7 gene mutations, comprises the steps of:
i. Realizing nucleic acid extraction from in vitro patient samples,
ii. Mixing the nucleic acids, obtained in step 1, with the reaction mixture comprising at least one or some or all of the delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6), Transv-target(wt) (SEQ ID NO: 7), Transv-target(m) (SEQ ID NO: 8) primers and optionally reaction buffer and/or sterile water and/or any DNA polymerase enzyme and/or Mg+2 ion, for instance, MgCl2 and/or more different enzyme or enzymes and/or more different reaction buffer and/or additional substance mixtures, or mixing the nucleic acids, obtained in step 1, with the reaction mixture comprising at least one of the delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6), Transv-target(wt) (SEQ ID NO: 7), Transv-target(m) (SEQ ID NO: 8) primers and/or delp (SEQ ID NO: 9), delPm (SEQ ID NO: 10), transvP (SEQ ID NO: 11), transvPm (SEQ ID NO: 12) probes and optionally reaction buffer and/or sterile water and/or any DNA polymerase enzyme and/or Mg+2 ion, for instance, MgCl2 and/or more different enzyme or enzymes and/or more different reaction buffer and/or additional substance mixtures, 10)
iii. Mixing the nucleic acids, obtained in step 1, with the reaction mixture comprising one or some or all of delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6) Transv-target(wt) (SEQ ID NO: 7), Transv-target(m) (SEQ ID NO: 8) primers or any primer and probe mixture or mixtures with a completely different array from the primer and prob arrays of which the sequence list is given above, primers and probe mixture or mixtures, primer and probe mixture or mixtures, primer and probes mixture or mixtures, or primers and probes mixture or mixtures or single primer or primers mixture or single probe or probes mixture with a completely different array from the primer and probe arrays of which the sequence list is given above and optionally reaction buffer and/or sterile water and/or any DNA polymerase enzyme and/or Mg+2 ion, for instance, MgCl2 and/or more different enzyme or enzymes and/or more different reaction buffer and/or additional substance mixtures,
iv. Realizing DNA/RNA amplification,
V. Examining the sample by means of fluorescent spectroscopy,
vi. Determining whether the samples, where fluorescent signal is observed, have TLR7 gene mutation or not.

### Examples

### Material and Method

Peripheral blood samples have been taken and examined in terms of TLR7 gene mutations. Nucleic acid extractions have been realized from peripheral blood samples according to Qiagen DNA isolation kit usage manual (Catalogue no: 69506). After the extracted nucleic acids are dissolved in 100 μl TE, the nucleic acid concentrations have been measured in Nanodrop spectrophotometer (Thermo Fisher Scientific).

### Primers

The primers have been designed with Primer3 bio-informatics tool such that Melting Curve Analysis will be made by means of SYBR Green. The primer arrays and the target regions are given in Table 1.

TABLE 1
Primer Arrays

| | |
|---|---|
| delF | 5′-TTAGAGGAATTAGACATCTCT-3′ |
| (SEQ ID NO: 1) | |
| delR | 5′- TGA AAG ATT TGA GCC CA -3′ |
| (SEQ ID NO: 2) | |
| TransvF | 5′- TCA AAT CAG GAG TCT GAC GA -3′ |
| (SEQ ID NO: 3) | |
| TransvR | 5′- AGG TGC ACA GAA ACC GAT TA -3′ |
| (SEQ ID NO: 4) | |
| Del-target | TTAGAGGAATTAGACATCTCTAAAAATTCCCTAA |
| (wt) | GTTTCTTGCCTTCTGGAGTTTTTGATGGTATGCC |
| (SEQ ID NO: 5) | TCCAAATCTAAAGAATCTCTCTTTGGCCAAAAAT |
| | GGGCTCAAATCTTTCA |
| Del-target (m) | TTAGAGGAATTAGACATCTCTAAAAATTCCCTAA |
| (SEQ ID NO: 6) | GTTTCTTTCTGGAGTTTTTGATGGTATGCCTCCA |
| | AATCTAAAGAATCTCTCTTTGGCCAAAAATGGGC |
| | TCAAATCTTTCA |
| Transv-target | TCAAATCAGGAGTCTGACGAAGTATTTTCTACAA |
| (wt) | GATGCCTTCCAGTTGCGATATCTGGATCTCAGCT |
| (SEQ ID NO: 7) | CAAATAAAATCCAGATGATCCAAAAGACCAGCTT |
| | CCCAGAAAATGTCCTCAACAATCTGAAGATGTTG |
| | CTTTTGCATCATAATCGGTTTCTGTGCACCT |
| Transv-target | TCAAATCAGGAGTCTGACGAAGTATTTTCTACAA |
| (m) | GATGCCTTCCAGTTTCGATATCTGGATCTCAGCT |
| (SEQ ID NO: 8) | CAAATAAAATCCAGATGATCCAAAAGACCAGCTT |
| | CCCAGAAAATGTCCTCAACAATCTGAAGATGTTG |
| | CTTTTGCATCATAATCGGTTTCTGTGCACCT |

### Detection

Detection study has been realized by means of the reaction mixture comprising the various reagents given below and specific primers with the mentioned concentrations.

The reaction mixture has one-step characteristic, and as the template sample, RNA or DNA can be used. For deletion detection and transversion detection in each patient sample, separate enzyme mixture, which comprises deletion specific primers, is used for deletion detection, and separate enzyme mixture, which comprises transversion specific primers, is used for transversion detection.

TABLE 1
Deletion detection

| | 1 test |
|---|---|
| Material | (μl) |
| RT Buffer | 4.00 |
| Reverse Transcriptase (200 u/μl) | 2.00 |
| HotStart Taq-DNA Polymerase (5 u/μl) | 1.00 |
| delF specific primer (10 μM) | 1.25 |
| delR specific primer (10 μM) | 1.25 |
| 10 mM dNTP mix | 0.4 |
| SYBR Green dye | 0.5 |
| Water, Molecular Biology Grade | 6.6 |
| RNA/DNA patient sample | 5 |
| 25 mM MgCl2 | 1.5 |

Total reaction volume is 25 μl.

TABLE 2
Transversion detection

| | 1 test |
|---|---|
| Material | (μl) |
| RT Buffer | 4.00 |
| Reverse Transcriptase (200 u/μl) | 2.00 |
| HotStart Taq-DNA Polymerase (5 u/μl) | 1.00 |
| transvF primer (10 μM) | 1.25 |
| transvR primer (10 μM) | 1.25 |
| 10 mM dNTP mix | 0.4 |
| SYBR Green dye | 0.5 |
| Water, Molecular Biology Grade | 6.6 |
| RNA/DNA patient sample | 5 |
| 25 mM MgCl2 | 1.5 |

Total reaction volume is 25 μl.

### Protocol for DNA Sample:

| | | |
|---|---|---|
| 95° C. 15 min | | |
| 95° C. 15 s | | |
| 55° C. 30 s | {close oversize brace} | 40 cycles |
| 72° C. 15 s | | |

Melt curve, 0.5 increase from 65° C. to 95° C.

### Protocol for RNA Sample:

| | | |
|---|---|---|
| 45° C. 20 min | | |
| 95° C. 15 min | | |
| 95° C. 15 s | {close oversize brace} | 40 cycles |
| 55° C. 30 s | | |
| 72° C. 15 s | | |

Melt curve, 0.5 increase from 65° C. to 95° C.

### Probes:

Taqqman probes have been designed for use in real-time PCR. Probe arrays have been given in Table 2.

TABLE 2
3. Probe sequences

| SEQ | Reporter | | Quenching |
|---|---|---|---|
| Numbers | Groups | Sequence Groups | Groups |
| delp | FAM | TAAGTTTCTTGCCTTCTGGAGTT | — |
| (SEQ ID | | | |
| NO: 9) | | | |
| delPm | HEX | ATTCCCTAAGTTTCTTTCTGGAG | — |
| (SEQ ID | | TTTTTG | |
| NO: 10) | | | |
| transvP | Cy5 | TGCCTTCCAGTTGCGATATCTGG | BHQ2 |
| (SEQ ID | | A | |
| NO: 11) | | | |
| transvPm | Cy5,5 | TGCCTTCCAGTTTCGATATCTGG | BHQ2 |
| (SEQ ID | | A | |
| NO: 12) | | | |

### Detection Application Version

The detection study has been realized by means of the reaction mixture comprising the various reagents given below and specific primers and probes with the mentioned concentrations.

The reaction mixture has one-step characteristic, and as the template sample, RNA or DNA can be used. For deletion detection and transversion detection in each patient sample, separate enzyme mixture, which comprises deletion specific primers and probes, is used for deletion detection, and separate enzyme mixture, which comprises transversion specific primers and probes, is used for transversion detection.

TABLE 4
Deletion and transversion detection

| Material | 1 test (μl) | 25 tests (μl) | 100 tests (μl) |
|---|---|---|---|
| RT Buffer | 4.00 | 100.00 | 400.00 |
| Reverse Transcriptase (200 u/μl) | 2.00 | 50.00 | 200.00 |
| HotStart Taq-DNA Polymerase (5 u/μl) | 1.00 | 25.00 | 100.00 |
| delF primer (10 μM) | 0.75 | 18.75 | 75.00 |
| delR primer (10 μM) | 0.75 | 18.75 | 75.00 |
| delP Probe (10 μM) | 0.50 | 12.50 | 50.00 |
| delPm Probe (10 μM) | 0.50 | 12.50 | 50.00 |
| transvF primer (10 μM) | 0.75 | 18.75 | 75.00 |
| transvR primer (10 μM) | 0.75 | 18.75 | 75.00 |
| transvP Probe (10 μM) | 0.50 | 12.50 | 50.00 |
| transvPm Probe (10 μM) | 0.50 | 12.50 | 50.00 |
| 50 mM MgCl2 | 1.50000 | 37.50 | 150.00 |
| 10 mM dNTP mix | 0.4 | 10.00 | 40.00 |
| Water, Molecular Biology Grade | 4.1 | 102.50 | 410.00 |
| Total | 18.00 | | |

| | | |
|---|---|---|
| DNA or RNA | 2 | |
| Reaction Volume | 20.00 | |
| Controls | T7 | positive, (wild type) plasmid |
| | | DNA |
| | T7m | positive, mutant plasmid DNA |
| | MBG | |
| | water | negative, no template control |

### Protocol for DNA or RNA Sample:

| | | |
|---|---|---|
| 95° C. 15 min | | |
| 95° C. 15 s | {close oversize brace} | 40 cycles |
| 63° C. 30 s | | |

### Alternative Protocol for DNA or RNA Sample (Table 5):

| | | | |
|---|---|---|---|
| 45° C. | 20 min | | Hold |
| 95° C. | 10 min | | Hold |
| 95° C. | 30 s | | |
| 60° C. | 30 s | {close oversize brace} | 45 cycles |
| 72° C. | 30 s | | |

In the known state of the art, for DNA and RNA samples, cycles are realized with temperatures and durations which can be determined by means of routine experiments by the specialized person, and DNA amplification procedure is completed.

Measurement is made in fluorescent spectrophotometer for the obtained samples, and it is decided that the samples, of which the fluorescent peak is given, have TLR7 mutation.

The protection scope of the present invention is set forth in the annexed claims and cannot be restricted to the illustrative disclosures given above, under the detailed description. It is because a person skilled in the relevant art can obviously produce similar embodiments under the light of the foregoing disclosures, without departing from the main principles of the present invention.

What is claimed is:

1. A diagnosis kit for usage in real-time PCR device for detection of normal TLR7 gene and variants of TLR7 gene (TLR7 gene mutations) which are rare and which affect sufficient functioning of the TLR7 gene, wherein diagnosis kit comprises a reaction mixture comprising at least one of the delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6), Transv-target(wt) (SEQ ID NO: 7), Transv-target(m) (SEQ ID NO: 8) as primers and/or delp (SEQ ID NO: 9), delPm (SEQ ID NO: 10), transvP (SEQ ID NO: 11), transvPm (SEQ ID NO: 12) as probes.

2. The diagnosis kit according to claim 1, wherein the reaction mixture comprises a reaction mixture comprising at least one of delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6), Transv-target(wt) (SEQ ID NO: 7), Transv-target(m) (SEQ ID NO: 8) as primers.

3. The diagnosis kit according to claim 1, wherein the reaction mixture comprises a reaction mixture comprising at least one of delF (SEQ ID NO: 1), delR (SEQ ID NO: 2), TransvF (SEQ ID NO: 3), TransvR (SEQ ID NO: 4) primers and/or delp (SEQ ID NO: 9), delPm (SEQ ID NO: 10), transvP (SEQ ID NO: 11), transvPm (SEQ ID NO: 12) as probes.

4. The diagnosis kit according to claim 1, wherein the reaction mixture comprises a reaction mixture comprising at least one of delp (SEQ ID NO: 9), delPm (SEQ ID NO: 10), transvP (SEQ ID NO: 11), transvPm (SEQ ID NO: 12) as probes.

5. The diagnosis kit according to claim 1, wherein the reaction mixture comprises a reaction mixture comprising at least one of Del-target(wt) (SEQ ID NO: 5), Del-target(m) (SEQ ID NO: 6), Transv-target(wt) (SEQ ID NO: 7), Transv-target(m) (SEQ ID NO: 8) primers and/or delp (SEQ ID NO: 9), delPm (SEQ ID NO: 10), transvP (SEQ ID NO: 11), transvPm (SEQ ID NO: 12) as probes.

6. The diagnosis kit according to claim 1, wherein the reaction mixture comprises delF (SEQ ID NO: 1), delR (SEQ ID NO: 2) as primers.

7. The diagnosis kit according to claim 1, wherein the reaction mixture comprises TransvF (SEQ ID NO: 3), TransvR (SEQ ID NO: 4) as primers.

8. The diagnosis kit according to claim 1, wherein the reaction mixture comprises a mixture of delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6), Transv-target(wt) (SEQ ID NO: 7), Transv-target(m) (SEQ ID NO: 8) as primers.

9. The diagnosis kit according to claim 1, wherein the reaction mixture comprises a mixture of delF (SEQ ID NO: 1), delR (SEQ ID NO: 2), TransvF (SEQ ID NO: 3), TransvR (SEQ ID NO: 4) as primers.

10. The diagnosis kit according to claim 1, wherein the reaction mixture comprises Del-target(wt) (SEQ ID NO: 5), Del-target(m) (SEQ ID NO: 6) as primers.

11. The diagnosis kit according to claim 1, wherein the reaction mixture comprises Transv-target(wt) (SEQ ID NO: 7), Transv-target(m) (SEQ ID NO: 8) as primers.

12. The diagnosis kit according to claim 1, wherein the reaction mixture comprises delF (SEQ ID NO: 1), delR (SEQ ID NO: 2), Del-target (wtSEQ ID NO: 5), Del-target(m) (SEQ ID NO: 6) as primers.

13. The diagnosis kit according to claim 1, wherein the reaction mixture comprises TransvF (SEQ ID NO: 3), TransvR (SEQ ID NO: 4), Transv-target(wt) (SEQ ID NO: 7), Transv-target(m) (SEQ ID NO: 8) as primers.

14. The diagnosis kit according to claim 1, wherein the subject matter diagnosis kit further comprises reaction buffer and/or sterile water and/or any DNA polymerase enzyme and/or Mg⁺² ion, for instance, MgCl₂.

15. The diagnosis kit according to claim 1, wherein the subject matter diagnosis kit further comprises reaction buffer and/or sterile water and/or any DNA polymerase enzyme and/or Mg⁺² ion, for instance, MgCl₂ and/or more different enzyme or enzymes and/or more different reaction buffer and/or additional substance mixtures.

16. The diagnosis kit according to claim 1 for usage in detection of normal TLR7 gene and also variants of TLR7 gene (TLR7 gene mutations) which are rare and which affect sufficient functioning of the TLR7 gene.

17. (canceled)

18. A combination of delp (SEQ ID NO: 9), delPm (SEQ ID NO: 10), transvP (SEQ ID NO: 11), and transvPm (SEQ ID NO: 12) as probes for usage in detection of TLR7 gene mutations.

19. A method for detection of normal TLR7 gene and also variants of TLR7 gene (TLR7 gene mutations) which are rare and which affect sufficient functioning of the TLR7 gene, said method comprises the steps of:
i. realizing nucleic acid extraction from in vitro patient samples,
ii. mixing the nucleic acids, obtained in step i, with the reaction mixture comprising at least one of the delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6), Transv-target(wt) (SEQ ID NO: 7), Transv-target(m) (SEQ ID NO: 8) as primers and/or (SEQ ID NO: 9), delPm (SEQ ID NO: 10), transvP (SEQ ID NO: 11), transvPm (SEQ ID NO: 12) as probes and optionally a reaction buffer and/or sterile water and/or any DNA polymerase enzyme and/or Mg+2 ion,
iii. mixing the nucleic acids, obtained in step i, with the reaction mixture comprising one of delF (SEQ ID NO: 1), delR (SEQ ID NO: 2), TransvF (SEQ ID NO: 3), TransvR (SEQ ID NO: 4), Del-target(wt) (SEQ ID NO: 5), Del-target(m) (SEQ ID NO: 6) Transv-target(wt) (SEQ ID NO: 7), Transv-target(m) (SEQ ID NO: 8) as primers and/or one or some or all of the delp (SEQ ID NO: 9), delPm (SEQ ID NO: 10), transvP (SEQ ID NO: 11), transvPm (SEQ ID NO: 12) as probes or any primer and probe mixture or mixtures with a completely different array from the primer and probe arrays of which the sequence list is given above, primers and probe mixture or mixtures, primer and probe mixture or mixtures, or primers and probes mixture or mixtures or single primer or primers mixture or single probe or probes mixture with a completely different array from the primer and probe arrays of which the sequence list is given above and optionally reaction buffer and/or sterile water and/or any DNA polymerase enzyme and/or Mg+2 ion,
iv. realizing DNA/RNA amplification,
v. examining the sample by means of fluorescent spectroscopy,
vi. determining whether the samples, where fluorescent signal is observed, have TLR7 gene mutation or not.
